(19) United States
(12) Patent Application Publication
(10) Pub. No.: US 2012/0248376 A1
(43) Pub. Date: Oct. 4, 2012

(54) Synthesis, Recharging and Processing of Hydrogen Storage Materials Using Supercritical Fluids

(75) Inventor: Sean McGrady, Fredericton (CA)

(73) Assignee: UNIVERSITY OF NEW BRUNSWICK, Fredericton (CA)

(21) Appl. No.: 13/466,946

(22) Filed: May 8, 2012

Related U.S. Application Data
(63) Continuation of application No. 11/721,975, filed on Oct. 5, 2009, now abandoned.
(60) Provisional application No. 60/636,549, filed on Dec. 17, 2004.

(30) Foreign Application Priority Data
Dec. 7, 2005 (CA) 2529427

(51) Int. Cl. C09K 3/00; C07C 9/08; C07C 9/06; C07C 43/04; C07C 43/12

(52) U.S. Cl. 252/364; 568/671; 568/683; 585/16

(57) ABSTRACT

Processes for synthesizing, recharging, reprocessing and chemical doping of hydrogen storage materials utilizing supercritical fluids. The processes include dissolution or suspension of the material in a supercritical fluid mixed with hydrogen.

### CROSS-REFERENCE TO RELATED APPLICATIONS

This application is a continuation of U.S. application Ser. No. 11/721,975, filed on Jun. 16, 2007 (currently pending), which is incorporated herein by reference in its entirety. U.S. application Ser. No. 11/721,975 is a national stage of International Application Number PCT/CA2005/001908, filed Dec. 16, 2005 (now expired), which is incorporated herein by reference in its entirety. International Application Number PCT/CA2005/001908 cites for priority Canadian Application Number 2,529,427, filed Dec. 7, 2005 (now abandoned). International Application Number PCT/CA2005/001908 cites for priority U.S. application Ser. No. 60/636,549, filed Dec. 17, 2004 (now expired).

### FIELD OF THE INVENTION

This invention relates generally to hydrogen storage materials and more specifically relates to the synthesizing, recharging, reprocessing and chemical doping of hydrogen storage materials using supercritical fluids.

### BACKGROUND OF THE INVENTION

Hydrogen storage materials or media (HSMs) are a class of chemical compounds containing hydrogen in a chemically or physically bound form. There is a particular current interest in HSMs for hydrogen storage applications and in particular, for hydrogen-powered vehicles for use in a ‘hydrogen economy’. This use requires an on-board source of hydrogen fuel. Hydrogen storage for transportation must operate within minimum volume and weight specifications, supply enough hydrogen for sufficient distance, charge/recharge near room temperature, and provide hydrogen at rates fast enough for fuel cell locomotion of automotive vehicles. Therefore, in order to create a useful on-board source of hydrogen fuel, an efficient method of storing the hydrogen is required.

Despite optimism over the last three decades, a hydrogen economy remains a utopian vision. The United States Department of Energy (US DOE) Basic Science group recently summarized the fundamental scientific challenges that must be met before a hydrogen economy becomes viable. In Basic Research Needs For The Hydrogen Economy, US DOE Report, May 2003, the following design criteria were identified for a viable HSM:
(1) High hydrogen storage capacity (min 6.5 wt % H).
(2) Low H₂ generation temperature (T_{dec} ideally around 60-120° C.).
(3) Favorable kinetics for H₂ adsorption/ desorption.
(4) Low cost.
(5) Low toxicity and low hazards.

Virtually all HSMs used in prior art technologies have been known for several decades, and yet none of them meet all five of the criteria listed above. For example, a number of alloys such as FeTi, Mg₂Ni and LaNi₅ satisfy criteria (2)-(5) but fail on criterion (1), containing only a few wt % hydrogen when fully loaded. Li₃BeH₇ reversibly stores 8.7% hydrogen by weight, but is highly toxic, thereby failing on criterion (5). Materials such as LiBH₄ and NaBH₄ react rapidly with water (hydrolysis) to release large amounts of hydrogen, but this process is chemically irreversible. Many other materials satisfy criteria (1), (2), (4) and (5), but not criterion (3).

### SUMMARY OF THE INVENTION

Accordingly, this invention relates to the use of a supercritical fluid in a process of synthesizing an inorganic hydrogen storage material wherein the supercritical fluid is used as the reaction medium where the synthesis takes place is disclosed. The hydrogen storage material is inorganic and can be a metal hydride.

In accordance with another aspect of this invention, the invention provides a process of recharging and reprocessing discharged inorganic hydrogen storage materials wherein a supercritical fluid is used as a reaction medium is disclosed.

In accordance with another aspect of this invention, the invention provides a process of synthesizing an inorganic hydrogen storage material comprising the steps of: providing a mixture of the substrates of an inorganic hydrogen storage material, hydrogen and a fluid medium; bringing the fluid medium to its supercritical phase; and recovering the hydrogen storage material from the supercritical fluid.

In accordance with another aspect of this invention, the invention provides a process of recharging and reprocessing discharged inorganic hydrogen storage materials comprising steps of: providing a mixture of the discharged inorganic

hydrogen storage material, hydrogen and a fluid medium; subjecting the mixture to a pressure and temperature sufficient to bring the fluid medium to its supercritical phase; recovering the hydrogen storage material from the supercritical fluid.

In accordance with another aspect of this invention, the invention relates to a process of synthesizing an inorganic hydrogen storage material comprising the steps of (a) providing substrates of an inorganic hydrogen storage material, hydrogen and a fluid medium; (b) bringing the fluid medium into its supercritical phase to form a supercritical fluid which entrains the hydrogen storage material and the hydrogen; (c) reacting the substrates of the inorganic hydrogen storage material with hydrogen to form the hydrogen storage material; and (d) recovering the hydrogen storage material from the supercritical fluid.

In accordance with another aspect of this invention, the invention relates to a process of synthesizing an inorganic hydrogen storage material comprising the steps of: (a) providing substrates of an inorganic hydrogen storage material, hydrogen and a fluid medium; (b) bringing the fluid medium into its near-critical phase to form a near-critical fluid which entrains the hydrogen storage material and the hydrogen; (c) reacting the substrates of the inorganic hydrogen storage material with hydrogen to form the hydrogen storage material; and (d) recovering the hydrogen storage material from the near-critical fluid.

In accordance with another aspect of this invention, the invention relates to a a process of recharging a discharged inorganic hydrogen storage material comprising the steps of: (a) providing a discharged inorganic hydrogen storage material, hydrogen, and a fluid medium; (b) bringing the fluid medium into its supercritical phase to form a supercritical fluid; (c) reacting the hydrogen with the uncharged inorganic hydrogen storage material whereby the hydrogen storage material is recharged; and (d) recovering the charged hydrogen storage material from the fluid medium.

### DETAILED DESCRIPTION OF THE INVENTION

The present invention involves the use of supercritical fluids (SCFs) for the synthesis, recharging, reprocessing and chemical doping of inorganic HSMs. SCFs can be used either as a neat material, or in combination with small amounts of conventional solvents, to effect dissolution or suspension of the discharged HSM and its subsequent rehydrogenation.

Processes for synthesizing HSMs are known in the prior art. As a general example of such a prior art process, the complex hexahydride Na₃AlH₆ can be prepared by the high-pressure, high-temperature processes described in Equations 1 and 2[1]:

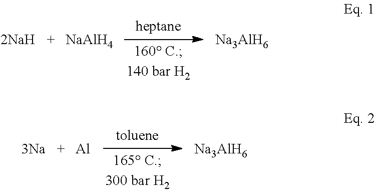

In one embodiment of the invention, the heptane and toluene in Eqs. 1 and 2 are replaced with supercritical ethane (T_{c} 32° C.; p_{c} 49 bar) or propane (T_{c} 97° C.; p_{c} 43 bar) which allows H₂ to become totally miscible with the organic solvent (the solvent is heptane in Eq. 1 and toluene in Eq. 2), and permits the reaction to occur at much lower temperatures (<120° C.) and hydrogen pressures (<50 bar) than for the reactions of Eq. 1 and 2.

This invention includes a process for bringing two reactants into contact and more particularly, H₂ and a hydrogen storage material. H₂ is generally very insoluble in organic solvents, such as heptane and toluene, requiring very high temperatures and hydrogen pressures to attain a significant concentration of dissolved H_{2.} Changing the reaction medium to a supercritical phase allows the H₂ to become totally miscible with the reaction medium, creating more favorable kinetics for the reaction. The heptane and toluene for Eqs. 1 and 2 set out above could be brought to their supercritical phases. Similarly, solvents used in hydrogenation reactions of the same general type as in Eqs. 1 and 2 set out above can be used in their supercritical phase. Preferably, the fluid medium should have a T_{c} that is higher than about room temperature and lower than about 120° C. Fluid media with a T_{c} above 120° C. can still be useful if the solvent properties are favourable to the reaction. For example, dimethyl ether, Me₂O has a rather high T_{c} of 127° C., but its polar nature and ability to coordinate to metal cations can make it the fluid medium of choice in certain instances. The T_{c} for heptane and toluene is 267 and 320° C., respectively. Any hydride that is stable above 150° C. would not be useful as an HSM, as it would require very high temperatures and unfavourable kinetics to discharge the hydrogen, therefore, not satisfying criteria (2) and (3) set out above. On the other hand, a hydride HSM with a discharge temperature equal to or lower than about room temperature would be more stable in its dehydrogenated state and therefore of no use as an HSM. This type of HSM would discharge too easily and would be very difficult to maintain charged. Preferred criteria for selection of an HSM according to the invention include a temperature window of SCF supercriticality and HSM reversibility that meets criteria (2) and (3) above, subject to compatible solvent-solute properties of the SCF and the HSM.

In another embodiment of the invention, the fluid medium is supercritical carbon dioxide (scCO₂) doped with small amounts of an ether solvent such as tetrahydrofuran (THF) or diethyl ether (Et₂O) which then dissolves/suspends a discharged inorganic HSM and exposes it efficiently for reaction with H₂. In a variation of this embodiment, a pure ether can also be used for this process. For example, dimethyl ether, Me₂O, becomes supercritical above 127° C. and 54 bar pressure. (Trifluoromethyl)methyl ether, CF₃OMe (T_{c}=134° C.) and methoxyethane, MeOEt (T_{c}=165° C.) can also be used as SCF media for this rehydrogenation.

The complete miscibility of H₂ with the SCF medium under the conditions of supercritical behavior according to the present invention ensures a fast and quantitative reaction to return the discharged HSM to its hydrogen-charged state. According to another embodiment of the invention the gas-solid kinetic issues that currently make it difficult to rehydrogenate NaAlH₄ are overcome through the use of a supercritical fluid as the reaction medium to synthesize and allow efficient reaction of the spent material with high concentrations of hydrogen (rehydrogenation of NaAlH₄ using prior art methods requires very severe conditions 200-400° C. and 100-400 bar H₂). In another embodiment of the invention, a dehydrogenated inorganic HSM is introduced into a pressure vessel or a SCF reactor, and pressurized at room temperature with H₂ (10-40 bar) and CO₂ (60-80 bar). The vessel is then heated to an appropriate temperature (usually 60-100° C.), to ensure that the mixture enters its supercritical phase. The mixture is stirred to ensure homogeneous temperature and composition throughout. The course of the reaction may be monitored spectroscopically by means of a window built into the body of the vessel, or by insertion of a fiber optic sensor through one of the walls. A small amount of a co-solvent, such as tetrahydrofuran (THF) or diethyl ether (Et₂O), may be added along with the dehydrogenated HSM when the vessel is loaded to assist dissolution of the dehydrogenated HSM and its efficient distribution throughout the SCF during the course of the reaction.

A further advantage afforded by this invention is the improved material properties achievable by rapid expansion of a supercritical solution (RESS) [2]. This produces a fine, dry powder with a large surface area and a narrow distribution of particle sizes—all desirable properties for a reproducible HSM. For example, RESS has found utility in the pharmaceutical industry for ensuring reliable drug delivery [3]. A second major drawback in recharging HSMs using heterogeneous gas-solid methods is the degradation and sintering of the solid during thermal cycling. Dissolution and reconstitution in an SCF medium each time it is recharged affords greater sample quality and reliability, and can be incorporated into a recharging technology much more easily than high-pressure hydrogenation. RESS can also be used as a method for introducing small amounts of a transition-metal catalyst into samples of an HSM from solution, rather than via mechanochemical methods [4].

In addition to NaAlH₄, other metal hydrides and their complexes, or mixtures thereof known to be useful as HSMs, can be synthesized, recharged and reprocessed using the methods of the invention. For example, KAlH₄, Mg(AlH₄)₂ and Ca(AlH₄)₂ can also be synthesized, recharged and reprocessed using the methods of the invention.

In another embodiment of this invention, two distinct HSMs can be co-dissolved and co-precipitated from a SCF, thereby producing a novel HSM with improved properties. For example, NaAlH₄ and KAlH₄ can be formed into a solid solution. Although KAlH₄ contains a heavier alkali metal cation, and consequently has a lower wt % hydrogen than its Na congener, it decomposes smoothly and reversibly without the assistance of a transition metal catalyst. Thus, a 2:1 mixture of the sodium and potassium alanates deposited by RESS from a supercritical solution will produce Na₂KAlH₆, (6.8 wt % H), with superior H₂ adsorption/desorption characteristics to pure NaAlH₄.

In a further embodiment of this invention, an expanded solvent or near-critical fluid may be used as the reaction medium in place of a SCF. Expanded solvents are conventional solvents that have been pressurized with a gas such as CO₂, to pressures below p_{c}[5]. A typical example of an expanded solvent is acetonitrile (CH₃CN) pressurized with around 50 bar CO₂, to give a CH₃CN/CO₂ mixture of approximate composition 1:2. The solubility of a permanent gas like H₂ is typically two orders of magnitude greater in such an expanded solvent than it is in the conventional solvent; at the same time, the high proportion of solvent present ensures that the medium is able to solvate the substrate effectively. Near-critical fluids are fluids close to but below either T_{c} or p_{c}; there are dramatic changes in the density, dielectric constant and solvent power as the critical point is approached. For the purposes of this invention the fluid will become less dense and its capacity to dissolve H₂ will increase rapidly in the near-critical regime.

### REFERENCES

[1] Zakharkin, L. I.; Gavrilenko, V. V. Dokl. Akad. Nauk. SSSR 1962 145, 793. Ashby, E. C.; Kobetz, P. Inorg. Chem. 1966, 5, 1615.
[2] Darr, J. A.; Poliakoff, M. Chem. Rev. 1999, 99, 495.
[3] Sievers, R. E.; Hybertson, B. M.; Hansen, B. N. U.S. Pat. No. 5,301,664, 1994.
[4] (a) Bogdanović, B.; Schwickardi, M. J. Alloys Comp. 1997, 253-254, 1. (b) Bogdanović, B.; Schwickardi, M. U.S. Pat. No. 6,814,782, 2004.
[5] (a) Musie, G.; Wei, M.; Subramanim, B.; Busch, D. H. Coord. Chem. Rev, 2001, 219-221, 789. (b) Wei, M.; Muise, G. T.; Busch, D. H.; Subramaniam, B. J. Am. Chem. Soc. 2002, 124, 2513.

I claim:

1. Use of a supercritical fluid in a process of synthesizing an inorganic hydrogen storage material wherein the supercritical fluid is used as a reaction medium where the synthesis takes place.
